(19) United States
(12) Patent Application Publication
(10) Pub. No.: US 2024/0197822 A1
(43) Pub. Date: Jun. 20, 2024

(54) USE OF EXTENSIVELY HYDROLYSED PROTEIN

(71) Applicants: MJN U.S. Holdings LLC, Evansville, IN (US); The General Hospital Corporation, Boston, MA (US)

(72) Inventors: Alessio FASANO, Boston, MA (US); Gabriele GROSS, Nijmegen (NL); Sarmauli Irianti MANURUNG, Bergen (NL); Stefania SENGER, Boston, MA (US); Eric Alexander Franciscus VAN TOL, Zeist (NL)

(21) Appl. No.: 18/572,580

(22) PCT Filed: Jun. 24, 2022
(86) PCT No.: PCT/EP2022/067380

§ 371 Date: Dec. 20, 2023

(30) Foreign Application Priority Data
Jun. 25, 2021 (GB) 2109176.4

(51) Int. Cl. A61K 38/17; A61P 29/00

(52) U.S. Cl. CPC A61K 38/1709; A61P 29/00

(57) ABSTRACT

The present application relates to extensively hydrolysed protein, and compositions comprising extensively hydrolysed protein, for use in reducing the risk of intestinal inflammation, reducing the risk of intestinal tissue damage, and/or improving gut barrier function, in an infant.

### FIELD OF THE INVENTION

The present application relates to extensively hydrolysed protein, and compositions comprising extensively hydrolysed protein, for use in reducing the risk of intestinal inflammation, reducing the risk of intestinal tissue damage, and/or improving gut barrier function, in an infant. More specifically, the present application is directed to extensively hydrolysed protein, and compositions comprising extensively hydrolysed protein, for use in improving gut barrier function and/or reducing the risk of intestinal inflammation, in a preterm infant.

### BACKGROUND

In the human body, the intestinal epithelium forms a barrier that separates the external environment (i.e. the contents of the intestinal lumen) and the body. Typically, the intestinal epithelium of preterm infants is less well developed than the intestinal epithelium of full-term infants. Consequently, the microbe-related pattern recognition process in preterm infants is immature, compared to that of full-term infants. As a result, the presence of commensal bacteria in the gut of preterm infants, which a full-term infant can usually tolerate with little or no significant side effects, can lead to excessive inflammatory responses and thus, excessive intestinal inflammation.

Gut barrier function refers to the function of the intestinal epithelium to allow passage of desired nutrients through the intestinal epithelium to the rest of the body, but to prevent potentially harmful substances (e.g. antigens) from leaving the intestine. Excessive intestinal inflammation in preterm infants can reduce gut barrier function as it has been shown to reduce gut barrier integrity, which often results in an undesirable increase in intestinal permeability. Excessive intestinal inflammation in preterm infants can also reduce gut barrier function as it is known to increase intestinal epithelial cytotoxicity (i.e. decrease foetal epithelial cell viability), which can also reduce gut barrier integrity. Excessive intestinal inflammation in preterm infants can therefore lead to the development of systemic infection and/or necrotising enterocolitis (Mihai et al., J. Pediatr., 2019).

The expression of cytokines, such as interleukin 1 beta (IL-1β), interleukin 8 (IL-8), and the tumour necrosis factor (TNF), is upregulated in instances of excessive intestinal inflammation. TNF is known to exert adverse effects on the intestinal epithelium by promoting cell death. These effects of TNF are partly mediated by signalling pathways via the TNF-receptor 1 (TNFR1) and/or TNF-receptor 2 (TNFR2). In addition, in certain cell contexts, cytokines, such as IL-1β, IL-8, and TNF, act as pro-inflammatory mediators. Therefore, these cytokines are useful indicators/markers of (excessive) intestinal inflammation in preterm infants.

A mother's milk is instrumental in promoting intestinal maturation and function in their infant, so as to reduce or counteract the negative effects of an immature microbe-related pattern recognition process. Mother's milk therefore plays a crucial role in protecting infants from (the effects of) excessive intestinal inflammation. When an infant is born prematurely, it is not always possible for the infant to receive their mother's milk. In these situations, an alternative method of providing such protective effects is employed, wherein the infant is provided with functional ingredients in the form of a preterm infant formula, or as a supplement for donor's milk e.g. a human milk fortifier.

Accordingly, there exists a need for functional infant formula ingredients that are capable of exerting a protective effect on the gut of a preterm infant. It would be particularly advantageous if the protective effect exerted by the functional formula ingredient is the reduction in the risk of intestinal inflammation, the reduction in the risk of intestinal tissue damage, and/or the improvement of gut barrier function, in a premature infant.

### SUMMARY OF INVENTION

In a first aspect, there is provided extensively hydrolysed casein for use in reducing the risk of intestinal inflammation, intestinal tissue damage, or both, in a preterm infant.

Preferably, reducing the risk of intestinal inflammation, intestinal tissue damage, or both, in a preterm infant, comprises a decrease in the expression of one or more of IL-1β, IL-8, and TNFR2, in the intestinal epithelium of the preterm infant, by 1% to 100%, compared to the absence of extensively hydrolysed casein.

Preferably, reducing the risk of intestinal inflammation, intestinal tissue damage, or both, in a preterm infant, comprises reducing the risk of necrotising enterocolitis (NEC), systemic infection, or both.

In a second aspect, there is provided extensively hydrolysed casein for use in improving gut barrier function in a preterm infant.

Preferably, improving gut barrier function in a preterm infant comprises improving intestinal cell viability in the preterm infant.

Alternatively, improving gut barrier function in a preterm infant comprises improving gut barrier integrity in the preterm infant.

Preferably, improving gut barrier integrity in a preterm infant comprises an increase in gut barrier integrity of 1% to 100%, an increase in intestinal cell viability of 1% to 100%, or both, compared to the absence of extensively hydrolysed casein.

Alternatively, improving gut barrier integrity in a preterm infant comprises reducing intestinal permeability in the preterm infant. Preferably, reducing intestinal permeability comprises a decrease in gut barrier permeability of 1% to 100%, compared to the absence of extensively hydrolysed casein.

Alternatively, improving gut barrier integrity in a preterm infant comprises improving intestinal cell viability in the preterm infant. Preferably, improving intestinal cell viability comprises an increase in intestinal cell viability of 1% to 100%, compared to the absence of extensively hydrolysed casein.

In a third aspect, there is provided extensively hydrolysed casein for use in downregulating expression of IL-1β, IL-8, TNFR2, or any combination thereof, in a preterm infant.

Preferably, downregulating expression of IL-1β, IL-8, TNFR2, or any combination thereof comprises a decrease in the expression of one or more of IL-1β, IL-8, and TNFR2, in the intestinal epithelium of the preterm infant, by 1% to 100%, compared to the absence of extensively hydrolysed casein.

Preferably, the extensively hydrolysed casein of any of the first, second, or third aspects comprises at least three peptides selected from SEQ ID NO: 1 to SEQ ID NO: 68. More preferably, the extensively hydrolysed casein comprises at least three peptides selected from SEQ ID NO: 1 to SEQ ID NO: 12.

Preferably, the extensively hydrolysed casein of any of the first, second, or third aspects is in the form of a reconstituted solution. More preferably, the reconstituted solution comprises extensively hydrolysed casein in the range of about 0.01 milligrams per millilitre (mg/mL) to about 0.50 grams per millilitre (g/mL).

In a fourth aspect, there is provided a composition for use according to any of the first, second, or third aspects, wherein the composition comprises extensively hydrolysed casein.

Preferably, the extensively hydrolysed casein comprises at least three peptides selected from SEQ ID NO: 1 to SEQ ID NO: 68. More preferably, the extensively hydrolysed casein comprises at least three peptides selected from SEQ ID NO: 1 to SEQ ID NO: 12.

Preferably, the composition is in the form of a reconstitutable powder. More preferably wherein the reconstitutable powder comprises extensively hydrolysed casein in the range of about 10 micrograms per 100 kilocalories (ug/100 kcal) to about 15 grams per 100 kilocalories (g/100 kcal).

Preferably, the composition further comprises at least one prebiotic. More preferably, the at least one prebiotic comprises polydextrose, galactooligosaccharides, or a combination thereof.

Preferably, the composition further comprises milk fat globule membrane (MFGM), preferably the MFGM is provided by an enriched milk product.

Preferably, the composition is a nutritional composition.

Preferably, the composition is a preterm infant formula or a human milk fortifier.

Preferably, the composition is a synthetic composition.

In a fifth aspect, there is provided a method for reducing the risk of intestinal inflammation, intestinal tissue damage, or both, in a preterm infant, the method comprising the step of administering extensively hydrolysed casein to the preterm infant.

In a sixth aspect, there is provided a method for improving gut barrier function in a preterm infant, the method comprising the step of administering extensively hydrolysed casein to the preterm infant.

In a seventh aspect, there is provided a method for downregulating expression of IL-1β, IL-8, TNFR2, or any combination thereof, in a preterm infant, the method comprising the step of administering extensively hydrolysed casein to the preterm infant.

In an eighth aspect, there is provided a method for: reducing the risk of intestinal inflammation, intestinal tissue damage, or both; improving gut barrier function; and/or, downregulating expression of IL-1β, IL-8, TNFR2, or any combination thereof, in a preterm infant, the method comprising administering a composition comprising extensively hydrolysed casein to the preterm infant.

### Definitions

“Milk” means a substance that has been drawn or extracted from the mammary gland of a mammal.

“Milk-based composition” means a composition comprising any mammalian milk-derived or mammalian milk-based product known in the art. For example, a “milk-based composition” may comprise bovine casein, bovine whey, bovine lactose, bovine milk fat globule membrane, bovine milk fat, or any combination thereof.

“Enriched milk product” generally refers to a milk ingredient that has been enriched with milk fat globule membrane (MFGM) and/or certain MFGM components, such as proteins and lipids found in the MFGM.

“Nutritional composition” means a substance or composition that satisfies at least a portion of a subject's nutrient requirements. “Nutritional composition(s)” may refer to liquids, powders, solutions, gels, pastes, solids, concentrates, suspensions, ready-to-use forms of enteral formulas, oral formulas, formulas for infants, follow-up formulas, formulas for paediatric subjects, formulas for children, and/or young child milks.

“Reconstituted solution”, in terms of the present disclosure, means the solution prepared when a diluent (e.g. water, saline, etc.) is added to an ingredient (e.g. a powder, a solution, a gel, a suspension, a paste, a solid, a liquid, a liquid concentrate, etc.). When the term “reconstituted solution” is used in reference to extensively hydrolysed protein, this means the solution prepared by the addition of a diluent (e.g. water, saline, etc.) to a form of extensively hydrolysed protein/extensively hydrolysed protein-containing composition i.e. in the form of a powder, a solution, a gel, a suspension, a paste, a solid, a liquid, a liquid concentrate, etc.

The term “synthetic” when applied to a composition, a nutritional composition, or a mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Paediatric subject” means a human under 18 years of age. The term “adult”, in terms of the present disclosure, refers to a human that is 18 years of age or greater. The term “paediatric subject” may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject” refers to a human subject between 6 and 12 years of age.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37th week of gestation. “Full-term” means an infant born after the end of the 37th week of gestation.

“Child” means a subject ranging from 12 months to 13 years of age. A child may be a subject between the ages of 1 and 12 years old. In another aspect, the terms “children” or “child” may refer to subjects that are between 1 and about 6 years old. Alternatively, the terms “children” or “child” may refer to subjects that are between about 7 and about 12 years old. The term “young child” means a subject ranging from 1 year to 3 years of age.

“Infant formula” means a composition that satisfies at least a portion of the nutrient requirements of an infant.

“Follow-up formula” means a composition that satisfies at least a portion of the nutrient requirements of an infant from the 6th month onwards, and for young children from 1 to 3 years of age.

“Young child milk”, in terms of the present disclosure, means a fortified milk-based beverage intended for children over one year of age (typically from one to six years of age). Young child milks are designed with the intent to serve as a complement to a diverse diet, to provide additional insurance that a child achieves continual, daily intake of all essential vitamins and minerals, macronutrients plus additional functional dietary components, such as non-essential nutrients that have purported health-promoting properties.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

The term “human milk oligosaccharides” or “HMOs” refers generally to a number of complex carbohydrates found in human breast milk.

“Normal gut barrier function”, in terms of the present disclosure, describes when the intestinal epithelium functions as desired i.e. it allows absorption of a desired amount of nutrients into the body, it allows a desired absorption of water and/or solutes into the body, and/or it limits the passage of potentially harmful substances (e.g. antigens, pathogens), to the rest of the body.

The expression “reduced gut barrier function”, in terms of the present disclosure, refers to the situation where the intestinal epithelium does not function as optimally as desired i.e. it blocks the passage of a desired amount of nutrients through, it allows too much water and/or solutes through, and/or it allows the passage of potentially harmful substances (e.g. antigens), to the rest of the body.

The term “intestinal permeability” refers to the passage of material from the gastrointestinal tract, through the intestinal epithelium, to the systemic circulation and thereby the rest of the body. Passage through the intestinal epithelium typically occurs via a transepithelial/transcellular route (i.e. through epithelial cells) or a paracellular route (i.e. between epithelial cells). Paracellular passage is the predominant route of intestinal epithelium passage of water and solutes, with tight junctions being the most important regulators of paracellular passage.

The term “degree of hydrolysis” refers to the extent to which peptide bonds, within a protein, are broken by a hydrolysis method. The degree of protein hydrolysis for the purposes of characterising the hydrolysed protein component of the composition is easily determined by one of ordinary skill in the formulation arts, by quantifying the amino nitrogen to total nitrogen ratio (AN/TN) of the protein component of the selected composition. The amino nitrogen component is quantified by USP titration methods for determining amino nitrogen content, with the total nitrogen component being determined by the Kjeldahl method. These methods are well-known to one of ordinary skill in the analytical chemistry art.

The term “partially hydrolysed”, in terms of the present disclosure, means having a degree of hydrolysis which is greater than 0% but less than about 40%.

The term “extensively hydrolysed”, in terms of the present disclosure, means having a degree of hydrolysis which is greater than or equal to about 40%.

The term “peptide” describes linear molecular chains of amino acids, including single chain molecules or their fragments. The term “small peptide”, in terms of this disclosure, means a peptide comprising no more than 50 total amino acids. The small peptides of the present disclosure may be naturally occurring, result from protein hydrolysis, or they may be synthesised.

The term “substantially free” means containing less than a functional amount of the specified component, typically less than 0.1% by weight, and includes 0% by weight of the specified ingredient.

As applied to nutrients, the term “essential” refers to any nutrient that cannot be synthesised by the body in amounts sufficient for normal growth, so it must be supplied by the diet. The term “conditionally essential” as applied to nutrients means that the nutrient must be supplied by the diet when adequate amounts of the precursor compound is unavailable to the body for endogenous synthesis to occur.

The term “probiotic” refers to microorganisms, such as bacteria or yeast, which have been shown to exert a beneficial effect on the health of a host subject. Probiotics can usually be classified as ‘viable’ or ‘non-viable’. The term ‘viable probiotics’ refers to living microorganisms, with the amount of a viable probiotic being detailed in colony-forming units (CFU). Probiotics that have been heat-killed, or otherwise inactivated, are termed ‘non-viable probiotics’ i.e. non-living microorganisms. Non-viable probiotics may still retain the ability to favourably influence the health of the host even though they may have been heat-killed or otherwise inactivated.

The term “prebiotic” refers to a non-digestible food ingredient that beneficially affects the host by selectively stimulating the growth and/or activity of one or a limited number of bacteria in the digestive tract, which can improve the health of the host. Prebiotics exert health benefits, which may include, but are not limited to: selective stimulation of the growth and/or activity of one or a limited number of beneficial gut bacteria; stimulation of the growth and/or activity of ingested probiotic microorganisms; selective reduction in gut pathogens; and, favourable influence on gut short chain fatty acid profile. The prebiotic of the composition may be naturally-occurring, synthetic, or developed through the genetic manipulation of organisms and/or plants, whether such new source is now known or developed later.

The term “organism” refers to any contiguous living system, such as an animal, plant, fungus, or micro-organism.

“Non-human lactoferrin” refers to lactoferrin that is produced by or obtained from a source other than human breast milk.

The term “sialic acid” refers to a family of derivatives of neuraminic acid. N-acetylneuraminic acid (Neu5Ac) and N-glycolylneuraminic acid (Neu5Gc) are among the most abundant, naturally-found forms of sialic acid, especially Neu5Ac in human and cow's milk.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24 hour period.

All references to singular characteristics or limitations in the present disclosure shall include the corresponding plural characteristic or limitation, and vice versa, unless otherwise specified or clearly implied to the contrary, by the context in which the reference is made.

All combinations of method or process steps disclosed herein can be performed in any order, unless otherwise specified or clearly implied to the contrary, by the context in which the referenced combination is made.

The compositions of the present disclosure can comprise, consist of, or consist essentially of any of the components described herein, as well as including any additional useful component.

### DETAILED DESCRIPTION

The present invention provides a novel intervention for reducing the risk of intestinal inflammation, reducing the risk of intestinal tissue damage, and/or improving gut barrier function, in preterm infants, via the provision of extensively hydrolysed protein, and compositions comprising extensively hydrolysed protein.

The inventors have surprisingly found that extensively hydrolysed protein supported cell viability and modulated inflammatory markers in an in vitro foetal epithelial cell model. These findings suggest that extensively hydrolysed protein may reduce the risk of intestinal inflammation, reduce the risk of intestinal tissue damage, and/or improve gut barrier function, in preterm infants. As excessive intestinal inflammation can result in systemic infection (such as sepsis) and/or necrotising enterocolitis in preterm infants, extensively hydrolysed protein could potentially reduce the risk of systemic infection and/or necrotising enterocolitis in preterm infants. This was not previously known or suggested, and was unexpected.

Extensively hydrolysed protein was found to downregulate the expression of IL-1β, IL-8, and TNFR2 in premature intestinal epithelium (see ‘Experimental Procedure’ section). This suggests that extensively hydrolysed protein may be able to reduce, or even prevent, intestinal inflammation in a preterm infant.

Extensively hydrolysed protein was also found to reduce the paracellular permeability of the foetal intestinal epithelium, when compared to the control i.e. extensively hydrolysed protein strengthened the foetal intestinal epithelium and made it less susceptible to paracellular passage. This result demonstrates that extensively hydrolysed protein is able to improve preterm infant gut barrier integrity and thus, suggests that extensively hydrolysed protein is able to improve preterm infant gut barrier function.

Extensively hydrolysed protein was also shown to significantly reduce foetal epithelial cytotoxicity in response to either the presence of heat-killed commensal non-pathogenic human isolate E. coli bacteria (hereinafter “heat-killed commensal E. coli”), or a pro-inflammatory environment. This indicates that extensively hydrolysed protein is able to improve preterm infant gut cell viability in a challenged environment and thus, suggests that extensively hydrolysed protein is able to improve preterm infant gut barrier function and/or reduce the risk of intestinal tissue damage, in an inflammatory setting.

The inventors hypothesised that the observed protective effect of extensively hydrolysed protein, in the presence of heat-killed commensal E. coli, was due to an effect on TNF-mediated intestinal epithelial processes.

As well as being upregulated in instances of excessive inflammation, TNF has a number of important physiological effects in the intestinal epithelium, which include: acting as a negative regulator of intestinal tight junctions (i.e. increased TNF levels are correlated with an increase in intestinal permeability); promoting cell survival; or, promoting cell apoptosis, depending on the particular cell context (Coskun et al., BBA Mol. Basis of Disease, 2011). TNF has been shown to exert its disruptive effect on intestinal tight junctions either: (i) on the tight junction directly, via downregulation of zonula occludens-1 (ZO1) and/or occludin; or, (ii) through modulation of MLCK expression and consequent disruption of the actin cytoskeleton (Su et al., Gastroent, 2013).

Extensively hydrolysed protein was found to have no effect on the expression of ZO1 or occludin (data not shown), so it was concluded that the observed protective effect of extensively hydrolysed protein was not via route (i). Thus, route (ii) was investigated. The inventors unexpectedly found the extensively hydrolysed protein downregulated expression of TNFR2, but had no effect on TNFR1 or MLCK expression. It is therefore hypothesised that extensively hydrolysed protein exerts a protective effect on preterm infant intestinal epithelium via downregulation of TNFR2 expression, and the consequent quenching of the negative effects of TNF on MLCK expression and actin cytoskeleton structure.

Furthermore, the inventors surprisingly found that, unlike foetal epithelium, the presence of heat-killed commensal E. coli, in the absence of extensively hydrolysed protein, had no significant effect on TNFR2 expression in adult epithelium. This difference in TNFR2 expression, and subsequent effect of TNF on MLCK expression, may partly explain why preterm infant gut reacts differently to the presence of heat-killed commensal E. coli, compared to full-term infants and adults. The results of the in vitro foetal epithelial cell model therefore suggest that extensively hydrolysed protein may be a preterm infant-specific intervention for reducing the risk of intestinal inflammation, reducing the risk of intestinal tissue damage, and/or improving gut barrier function, in preterm infants. This was not previously known or suggested, and was unexpected.

The present invention therefore provides extensively hydrolysed protein, as well as providing novel compositions comprising extensively hydrolysed protein, for use in: reducing the risk of intestinal inflammation, intestinal tissue damage, or both; improving gut barrier function; downregulating expression of IL-1β, IL-8, TNFR2, or any combination thereof; or, any combination thereof, in a preterm infant.

The extensively hydrolysed protein of any aspect detailed below may comprise extensively hydrolysed casein, extensively hydrolysed whey, or a combination thereof. The extensively hydrolysed protein of any aspect detailed below may be derived from any mammalian animal milk protein or plant protein, as well as their fractions, or any combination thereof. The extensively hydrolysed protein may be derived from bovine milk, caprine milk, whey protein, casein protein, soy protein, rice protein, pea protein, peanut protein, egg protein, sesame protein, fish protein, wheat protein, hydrolysed protein, or any combination thereof. Bovine milk protein sources may comprise, but are not limited to, milk protein powders, milk protein concentrates, milk protein isolates, non-fat milk solids, non-fat milk, non-fat dry milk, whey protein, whey protein isolates, whey protein concentrates, sweet whey, acid whey, casein, acid casein, caseinate (e.g. sodium caseinate, sodium calcium caseinate, calcium caseinate), or any combination thereof.

The extensively hydrolysed protein of any aspect detailed below may comprise at least two peptides selected from the peptide sequences listed in Table 1, preferably three peptides, more preferably four peptides, even more preferably five peptides. In a preferred aspect, the extensively hydrolysed protein comprises at least two peptides selected from SEQ ID NO: 1 to SEQ ID NO: 12, preferably three peptides, more preferably four peptides, even more preferably five peptides. In another preferred aspect, the extensively hydrolysed protein comprises the following peptides from Table 1: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

TABLE 1

| | |
|---|---|
| SEQ ID NO: 1 | IPNPIG |
| SEQ ID NO: 2 | IGSESTEDQ |
| SEQ ID NO: 3 | DKTEIPT |
| SEQ ID NO: 4 | IVPN |
| SEQ ID NO: 5 | LEDSPE |
| SEQ ID NO: 6 | NQEQPI |
| SEQ ID NO: 7 | NVPGE |
| SEQ ID NO: 8 | PFPGPI |
| SEQ ID NO: 9 | TEDEL |
| SEQ ID NO: 10 | VPSE |
| SEQ ID NO: 11 | YPFPGP |
| SEQ ID NO: 12 | YPSGA |
| SEQ ID NO: 13 | FPGPIP |
| SEQ ID NO: 14 | MHQPHQPLPPT |
| SEQ ID NO: 15 | YPFPGPIPN |
| SEQ ID NO: 16 | DMEST |
| SEQ ID NO: 17 | FPGPIPN |
| SEQ ID NO: 18 | IPNPI |
| SEQ ID NO: 19 | MESTEV |
| SEQ ID NO: 20 | PGPIPN |
| SEQ ID NO: 21 | PHQPLPPT |
| SEQ ID NO: 22 | PNPI |
| SEQ ID NO: 23 | SKDIGSE |
| SEQ ID NO: 24 | YPFPGPIP |
| SEQ ID NO: 25 | AINPSKEN |
| SEQ ID NO: 26 | APFPE |
| SEQ ID NO: 27 | DIGSES |
| SEQ ID NO: 28 | DMPI |
| SEQ ID NO: 29 | DVPS |
| SEQ ID NO: 30 | EDI |
| SEQ ID NO: 31 | ELF |
| SEQ ID NO: 32 | EMP |
| SEQ ID NO: 33 | ETAPVPL |
| SEQ ID NO: 34 | GPFP |
| SEQ ID NO: 35 | GPIV |
| SEQ ID NO: 36 | IGSSSEES |
| SEQ ID NO: 37 | IGSSSEESA |
| SEQ ID NO: 38 | INPSKE |
| SEQ ID NO: 39 | IPPLTQTPV |
| SEQ ID NO: 40 | ITAP |
| SEQ ID NO: 41 | KHQGLPQ |
| SEQ ID NO: 42 | LDVTP |
| SEQ ID NO: 43 | LPLPL |
| SEQ ID NO: 44 | NAVPI |
| SEQ ID NO: 45 | NEVEA |
| SEQ ID NO: 46 | NLL |
| SEQ ID NO: 47 | PITPT |
| SEQ ID NO: 48 | PNSLPQ |
| SEQ ID NO: 49 | PQLEIVPN |
| SEQ ID NO: 50 | PQNIPPL |
| SEQ ID NO: 51 | PVLGPV |
| SEQ ID NO: 52 | PVPQ |
| SEQ ID NO: 53 | PVVVP |
| SEQ ID NO: 54 | PVVVPP |
| SEQ ID NO: 55 | SIGSSSEESAE |
| SEQ ID NO: 56 | SISSSEE |
| SEQ ID NO: 57 | SISSSEEIVPN |
| SEQ ID NO: 58 | SPPEIN |
| SEQ ID NO: 59 | SPPEINT |
| SEQ ID NO:60 | TDAPSFS |
| SEQ ID NO: 61 | VATEEV |
| SEQ ID NO: 62 | VLPVP |
| SEQ ID NO: 63 | VPGE |
| SEQ ID NO: 64 | VPGEIV |
| SEQ ID NO: 65 | VPITPT |
| SEQ ID NO: 66 | WVPPFLQPE |
| SEQ ID NO: 67 | WWVPP |
| SEQ ID NO: 68 | YPVEP |

The extensively hydrolysed protein of any aspect detailed below may be prepared by the following process. The particular protein is dissolved in water and the pH adjusted to between 5 to 9, preferably 6 to 8, via the addition of acid (such as hydrochloric acid, sulphuric acid, etc.) or base (such as, sodium hydroxide, potassium hydroxide, etc.). The protein is then hydrolysed via addition of at least one enzyme, to the solution, and the mixture left for as long as required to reach the desired degree of hydrolysis (e.g. ≥ 40% degree of hydrolysis); this may take 30 minutes or longer. The enzyme(s) used in the process could be mammalian proteases, microbial enzymes (e.g. Alcalase® from Novozymes, Protease NR from Amano Enzymes, FoodPro® Alkaline Protease from DuPont Nutrition & Biosciences, etc.), plant proteases (e.g. ficain), or a combination thereof. Before, during, and/or after enzyme addition, the reaction temperature is maintained between 30° C. and 65° C., preferably between 35° C. and 60° C. Once the desired degree of hydrolysis of the protein is achieved, the enzyme is then inactivated by heating the mixture to between 80° C. and 90° C. for 5 minutes. One or more separation steps, such as centrifugation, filtration (e.g. microfiltration, ultrafiltration, nanofiltration), or a combination thereof, may then be applied, if needed. The extensively hydrolysed protein can be used without further processing, or can be subjected to a final dehydration process, such as spray drying, freeze drying, or any other suitable dehydration process. Alternatively, the extensively hydrolysed protein of any aspect detailed below may be prepared by any suitable hydrolysis process known in the art.

In any aspect below, reducing the risk of intestinal inflammation and/or intestinal tissue damage may refer to a decrease in the expression of one or more of IL-1β, IL-8, and TNFR2, in the intestinal epithelium of a preterm infant, in the presence of extensively hydrolysed protein, of at least 1%, compared to a control value in the absence of extensively hydrolysed protein; preferably, a decrease in the expression of one or more of IL-1β, IL-8, and TNFR2 of at least 5%; more preferably, a decrease in the expression of one or more of IL-1β, IL-8, and TNFR2 of at least 10%; even more preferably, a decrease in the expression of one or more of IL-1β, IL-8, and TNFR2 of at least 20%; most preferably, a decrease in the expression of one or more of IL-1β, IL-8, and TNFR2 of at least 50%. Alternatively, in any aspect below, reducing the risk of intestinal inflammation and/or intestinal tissue damage may refer to a decrease in the expression of one or more of IL-1β, IL-8, and TNFR2, in the presence of extensively hydrolysed protein, of 1% to 100%, compared to a control value that is absent of extensively hydrolysed protein; preferably, a decrease in the expression of one or more of IL-1β, IL-8, and TNFR2 of 5% to 100%; more preferably, a decrease in the expression of one or more of IL-1β, IL-8, and TNFR2 of 10% to 100%. Alternatively, in any aspect below, improving reducing the risk of intestinal inflammation and/or intestinal tissue damage may refer to the restoration of the expression of one or more of IL-1β, IL-8, and TNFR2 in the intestinal epithelium of a preterm infant to close to the expression level(s) of a full-term infant; preferably, the expression level(s) are restored to that of a full-term infant; more preferably, the expression level(s) are lower than that of a full-term infant. The expression of one or more of IL-1β, IL-8, and TNFR2 may be quantified by the method detailed in Study 1 or Study 2a, or any suitable method for quantifying cytokine expression known in the art.

In any aspect below, improving gut barrier function may refer to an increase in gut barrier integrity of a preterm infant, in the presence of extensively hydrolysed protein, of at least 1%, compared to a control value in the absence of extensively hydrolysed protein; preferably, an increase in gut barrier integrity of at least 5%; more preferably, an increase in gut barrier integrity of at least 10%; even more preferably, an increase in gut barrier integrity of at least 20%; most preferably, an increase in gut barrier integrity of at least 50%. Alternatively, in any aspect below, improving gut barrier function may refer to an increase in gut barrier integrity, in the presence of extensively hydrolysed protein, of 1% to 100%, compared to a control value that is absent of extensively hydrolysed protein; preferably, an increase in gut barrier integrity of 5% to 100%; more preferably, an increase in gut barrier integrity of 10% to 100%. Alternatively, in any aspect below, improving gut barrier integrity may refer to the restoration of gut barrier integrity of a preterm infant to close to the gut barrier integrity of a full-term infant; preferably, the gut barrier integrity is restored to that of a full-term infant; more preferably, the gut barrier integrity is higher than that of a full-term infant. Gut barrier integrity may be quantified by the method detailed in Study 1, or any suitable method for quantifying gut barrier integrity known in the art.

In any aspect below, an increase in gut barrier integrity may refer to a decrease in intestinal permeability of a preterm infant, in the presence of extensively hydrolysed protein, of at least 1%, compared to a control value in the absence of extensively hydrolysed protein; preferably, a decrease in intestinal permeability of at least 5%; more preferably, a decrease in intestinal permeability of at least 10%; even more preferably, a decrease in intestinal permeability of at least 20%; most preferably, an increase in gut barrier integrity of at least 50%. Alternatively, in any aspect below, an increase in gut barrier integrity may refer to a decrease in intestinal permeability, in the presence of extensively hydrolysed protein, of 1% to 100%, compared to a control value that is absent of extensively hydrolysed protein; preferably, a decrease in intestinal permeability of 5% to 100%; more preferably, a decrease in intestinal permeability of 10% to 100%. Alternatively, in any aspect below, an increase in gut barrier integrity may refer to the restoration of intestinal permeability of a preterm infant to close to the intestinal permeability of a full-term infant; preferably, the intestinal permeability is restored to that of a full-term infant; more preferably, the intestinal permeability is lower than that of a full-term infant. Intestinal permeability may be quantified by the method detailed in Study 1, or any suitable method for quantifying intestinal permeability known in the art.

In any aspect below, improving gut barrier function may refer to an increase in gut barrier cell viability of a preterm infant, in the presence of extensively hydrolysed protein, of at least 1%, compared to a control value in the absence of extensively hydrolysed protein; preferably, an increase in gut barrier cell viability of at least 5%; more preferably, an increase in gut barrier cell viability of at least 10%; even more preferably, an increase in gut barrier cell viability of at least 20%; most preferably, an increase in gut barrier cell viability of at least 50%. Alternatively, in any aspect below, improving gut barrier function may refer to an increase in gut barrier cell viability, in the presence of extensively hydrolysed protein, of 1% to 100%, compared to a control value that is absent of extensively hydrolysed protein; preferably, an increase in gut barrier cell viability of 5% to 100%; more preferably, an increase in gut barrier cell viability of 10% to 100%. Alternatively, in any aspect below, improving gut barrier cell viability may refer to the restoration of gut barrier cell viability of a preterm infant to close to the gut barrier cell viability of a full-term infant; preferably, the gut barrier cell viability is restored to that of a full-term infant; more preferably, the gut barrier cell viability is higher than that of a full-term infant. Gut barrier cell viability may be quantified by the method detailed in Study 2a or 2b, or any suitable method for quantifying cell viability known in the art.

In any aspect below, wherein downregulating expression of IL-1β, IL-8, TNFR2, or any combination thereof may refer to a decrease in the expression of one or more of IL-1β, IL-8, and TNFR2, in the intestinal epithelium of a preterm infant, in the presence of extensively hydrolysed protein, of at least 1%, compared to a control value in the absence of extensively hydrolysed protein; preferably, a decrease in the expression of one or more of IL-1β, IL-8, and TNFR2 of at least 5%; more preferably, a decrease in the expression of one or more of IL-1β, IL-8, and TNFR2 of at least 10%; even more preferably, a decrease in the expression of one or more of IL-1β, IL-8, and TNFR2 of at least 20%; most preferably, a decrease in the expression of one or more of IL-1β, IL-8, and TNFR2 of at least 50%. Alternatively, in any aspect below, downregulating expression of IL-1β, IL-8, TNFR2, or any combination thereof may refer to a decrease in the expression of one or more of IL-1β, IL-8, and TNFR2, in the presence of extensively hydrolysed protein, of 1% to 100%, compared to a control value that is absent of extensively hydrolysed protein; preferably, a decrease in the expression of one or more of IL-1β, IL-8, and TNFR2 of 5% to 100%; more preferably, a decrease in the expression of one or more of IL-1β, IL-8, and TNFR2 of 10% to 100%. Alternatively, in any aspect below, downregulating expression of IL-1β, IL-8, TNFR2, or any combination thereof may refer to the restoration of the expression of one or more of IL-1β, IL-8, and TNFR2 in the intestinal epithelium of a preterm infant to close to the expression level(s) of a full-term infant; preferably, the expression level(s) are restored to that of a full-term infant; more preferably, the expression level(s) are lower than that of a full-term infant. The expression of one or more of IL-1β, IL-8, and TNFR2 may be quantified by the method detailed in Study 1 or Study 2a, or any suitable method for quantifying cytokine expression known in the art.

In one aspect, the present invention provides extensively hydrolysed protein for use in: reducing the risk of intestinal inflammation, intestinal tissue damage, or both; improving gut barrier function; downregulating expression of IL-1β, IL-8, TNFR2, or any combination thereof; or, any combination thereof, in a preterm infant. The extensively hydrolysed protein may be administered to the subject in the form of a powder, a solution, a gel, a suspension, a paste, a solid, a liquid, a liquid concentrate, or a reconstituted solution. Preferably, the extensively hydrolysed protein is administered to the subject in the form of a powder, a liquid concentrate, or a reconstituted solution. More preferably, the extensively hydrolysed protein is administered to the subject in the form of a reconstituted solution.

The extensively hydrolysed protein daily dosage may be varied depending on the requirement of the infant and the particular form of extensively hydrolysed protein. The daily dosage of extensively hydrolysed protein may be in the range of about 0.05 milligram per day (mg/day) to about 50 grams per day (g/day). Preferably, the daily dosage of extensively hydrolysed protein is in the range of about 0.1 mg/day to about 30 g/day. More preferably, the daily dosage of extensively hydrolysed protein is in the range of about 0.15 mg/day to about 25 g/day. Even more preferably, the daily dosage of extensively hydrolysed protein is in the range of about 0.2 mg/day to about 20 g/day. The dose of extensively hydrolysed protein may be in the form of a single daily dosage. Alternatively, the total daily dosage may be administered in portions throughout the day e.g. two portions, three portions, etc.

The extensively hydrolysed protein may be administered to the subject in an amount suitable to reduce the risk of intestinal inflammation, reduce the risk intestinal tissue damage, and/or improve gut barrier function. The extensively hydrolysed protein may be administered as a reconstituted solution comprising extensively hydrolysed protein in the range of about 0.01 milligrams per millilitre (mg/mL) to about 0.50 grams per millilitre (g/mL). Preferably, the reconstituted solution comprises extensively hydrolysed protein in the range of about 0.1 mg/mL to about 0.25 g/mL. More preferably, the reconstituted solution comprises extensively hydrolysed protein in the range of about 0.1 mg/mL to about 200 mg/mL. Even more preferably, the reconstituted solution comprises extensively hydrolysed protein in the range of about 0.1 mg/mL to about 100 mg/mL.

Alternatively, the extensively hydrolysed protein may be administered as a reconstituted solution comprising extensively hydrolysed protein in the range of about 10 micrograms per 100 kilocalories (μg/100 kcal) to about 25 grams per 100 kilocalories (g/100 kcal). Preferably, the reconstituted solution comprises extensively hydrolysed protein in the range of about 15 μg/100 kcal to about 20 g/100 kcal. More preferably, the reconstituted solution comprises extensively hydrolysed protein in the range of about 20 μg/100 kcal to about 15 g/100 kcal.

Alternatively, the extensively hydrolysed protein may be administered as a reconstituted solution comprising extensively hydrolysed protein in an amount of about 0.01% weight per volume (% w/v) to about 50% w/v. Preferably, the reconstituted solution comprises extensively hydrolysed protein in the range of about 0.1% w/v to about 20% w/v. More preferably, the reconstituted solution comprises extensively hydrolysed protein in the range of about 0.1% w/v to about 10% w/V.

In another aspect, the present invention provides a composition for use in: reducing the risk of intestinal inflammation, intestinal tissue damage, or both; improving gut barrier function; downregulating expression of IL-1β, IL-8, TNFR2, or any combination thereof; or, any combination thereof, in a preterm infant, wherein the composition comprises extensively hydrolysed protein.

The extensively hydrolysed protein may be present in the composition in an amount of at least about 10 μg/100 kcal. The composition may comprise extensively hydrolysed protein in the range of about 10 μg/100 kcal to about 25 g/100 kcal. Preferably, the composition comprises extensively hydrolysed protein in the range of about 15 μg/100 kcal to about 20 g/100 kcal. More preferably, the composition comprises extensively hydrolysed protein in the range of about 20 μg/100 kcal to about 15 g/100 kcal. The composition may be specifically designed for a paediatric subject. When the composition is specifically intended for a paediatric subject, the composition may comprise extensively hydrolysed protein in the range of about 10 μg/100 kcal to about 15 g/100 kcal. Preferably, the composition comprises extensively hydrolysed protein in the range of about 15 μg/100 kcal to about 12 g/100 kcal. More preferably, the composition comprises extensively hydrolysed protein in the range of about 15 μg/100 kcal to about 10 g/100 kcal.

The composition may be provided in any form known in the art. The composition may be provided in the form of a powder, a gel, a suspension, a paste, a solid, a liquid, a liquid concentrate, a reconstitutable powder, a reconstituted solution, or a ready-to-use product. Preferably, the composition is in the form of a reconstitutable powder, a reconstituted solution, or a ready-to-use product. Most preferably, the composition is provided in the form of a reconstitutable powder.

The composition may comprise a protein source, a fat or lipid source, a carbohydrate source, or any combination thereof. The composition may comprise one or more: probiotics; prebiotics; source of long-chain polyunsaturated fatty acids (LCPUFAs); human milk oligosaccharides (HMOS); β-glucan; sialic acid; suitable composition ingredient; or, any combination thereof.

The composition may comprise at least one protein source, in addition to the extensively hydrolysed protein, wherein the protein source provides protein to the composition. The protein source may comprise intact protein, partially hydrolysed protein, extensively hydrolysed protein, small amino acid peptides, or any combination thereof. The protein source may be derived from any mammalian animal milk protein or plant protein, as well as their fractions, or any combination thereof. The protein source may comprise bovine milk, caprine milk, whey protein, casein protein, soy protein, rice protein, pea protein, peanut protein, egg protein, sesame protein, fish protein, wheat protein, hydrolysed protein, or any combination thereof. Bovine milk protein sources may comprise, but are not limited to, milk protein powders, milk protein concentrates, milk protein isolates, non-fat milk solids, non-fat milk, non-fat dry milk, whey protein, whey protein isolates, whey protein concentrates, sweet whey, acid whey, casein, acid casein, caseinate (e.g. sodium caseinate, sodium calcium caseinate, calcium caseinate), or any combination thereof.

The composition may comprise a protein source in the range of about 1 g/100 kcal to about 7 g/100 kcal. Preferably, the composition comprises a protein source in the range of about 3.5 g/100 kcal to about 4.5 g/100 kcal. The protein source may comprise from about 40% to about 85% whey protein and from about 15% to about 60% casein.

As noted above, the protein source may comprise a source of intact protein. The composition may comprise intact protein in the range of about 1 g/100 kcal to about 3 g/100 kcal. Preferably, the composition comprises intact protein in the range of about 1 g/100 kcal to about 2.5 g/100 kcal. More preferably, the composition comprises intact protein in the range of about 1.3 g/100 kcal to about 2.1 g/100 kcal. The protein source may comprise a combination of intact protein and partially hydrolysed protein, wherein the partially hydrolysed protein may have a degree of hydrolysis of between about 4% and 10%.

As also noted above, the protein source of the composition may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. The proteins may be hydrolysed by any method known in the art. The terms “protein hydrolysates” or “hydrolysed protein” are used interchangeably herein and refer to hydrolysed proteins, wherein the degree of hydrolysis may be from about 20% to about 80%, or from about 30% to about 80%, or even from about 40% to about 60%.

The composition may comprise free amino acids. The free amino acids may comprise histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine, or any combination thereof. The free amino acids may be branched chain free amino acids. The amount of free amino acids in the composition may vary from about 1 g/100 kcal to about 5 g/100 kcal. The free amino acids may all have a molecular weight of less than 500 Da.

The composition may comprise an enriched milk product. The enriched milk product may be formed by fractionation of non-human (e.g. bovine) milk. The enriched milk product may have a total protein level in a range of between 20% and 90%; preferably, the enriched milk product has a total protein level in a range of between 65% and 80%.

The enriched milk product may comprise an enriched whey protein concentrate (eWPC). Alternatively, the enriched milk product may comprise an enriched lipid fraction derived from milk. The eWPC and the enriched lipid fraction may be produced by any number of fractionation techniques well known in the art. These techniques include, but are not limited to, membrane filtration, melting point fractionation, organic solvent fractionation, super critical fluid fractionation, or any combination thereof. Alternatively, eWPC is available commercially, including under the trade names Lacprodan MFGM-10 and Lacprodan PL-20, both available from Arla Food Ingredients of Viby, Denmark. With the addition of eWPC, the lipid composition of the composition can more closely resemble that of human milk.

The composition may comprise eWPC in the range of about 0.5 g/L to about 10 g/L. Preferably, the composition comprises eWPC in the range of about 1 g/L to about 9 g/L. More preferably, the composition comprises eWPC in the range of about 3 g/L to about 8 g/L. Alternatively, the composition may comprise eWPC in the range of about 0.06 g/100 kcal to about 1.5 g/100 kcal. Preferably, the composition comprises eWPC in the range of about 0.3 g/100 kcal to about 1.4 g/100 kcal. More preferably, the composition comprises eWPC in the range of about 0.4 g/100 kcal to about 1 g/100 kcal.

The composition may comprise at least one fat or lipid source, wherein the fat or lipid source provides fat and/or lipid to the composition. Suitable fat or lipid sources for the composition may be any known or used in the art. The fat or lipid source may be present in the composition in addition to another fat or lipid source, such as a LCPUFA. The fat or lipid source may comprise animal sources, such as milk fat, butter, butter fat, or egg yolk lipid; marine sources, such as fish oils, marine oils, or single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, or wheat germ oil; medium chain triglyceride oils; emulsions and esters of fatty acids; or any combination thereof.

The composition may comprise a fat or lipid source in the range of about 1 g/100 kcal to about 10 g/100 kcal. Preferably, the composition comprises a fat or lipid source in the range of about 2 g/100 kcal to about 7 g/100 kcal of a fat or lipid source. More preferably the composition comprises a fat or lipid source in the range of about 2.5 g/100 kcal to about 6 g/100 kcal. Most preferably, the composition comprises a fat or lipid source in the range of about 3 g/100 kcal to about 4 g/100 kcal.

The composition may comprise at least one carbohydrate source, wherein the carbohydrate source provides carbohydrate to the composition. The carbohydrate source may be present in the composition in addition to another carbohydrate source, such as PDX and GOS. The carbohydrate source may comprise lactose, glucose, fructose, sucrose, starch, maltodextrin, maltose, fructooligosaccharides, corn syrup, high fructose corn syrup, dextrose, corn syrup solids, rice syrup solids, or any combination thereof. Moreover, hydrolysed, partially hydrolysed, and/or extensively hydrolysed carbohydrates may be desirable for inclusion in the composition due to their easy digestibility. More specifically, hydrolysed carbohydrates are less likely to contain allergenic epitopes. The composition may therefore comprise a carbohydrate source comprising hydrolysed or intact, naturally or chemically modified, starches sourced from corn, tapioca, rice, or potato, in waxy or non-waxy forms, such as hydrolysed corn starch.

The composition may comprise a carbohydrate source in the range of about 5 g/100 kcal to about 25 g/100 kcal. Preferably, the composition comprises a carbohydrate source in the range of about 6 g/100 kcal to about 22 g/100 kcal. More preferably, the composition comprises a carbohydrate source in the range of about 12 g/100 kcal to about 14 g/100 kcal.

The composition may comprise one or more probiotics. The probiotic may comprise any Bifidobacterium species, any Lactobacillus species, or a combination thereof. Preferably, the probiotic is Bifidobacterium adolescentis (ATCC number 15703), Bifidobacterium animalis subsp. lactis, Bifidobacterium breve, Bifidobacterium longum subsp. infantis (B. infantis), Lactobacillus acidophilus, Lactobacillus gasseri (ATCC number 33323), Lactobacillus reuteri (DSM number 17938), Lactobacillus rhamnosus GG (LGG; ATCC number 53103), or any combination thereof. More preferably, the probiotic is LGG, B. infantis, or a combination thereof.

The probiotic may be viable or non-viable. The probiotic incorporated into the composition may comprise both viable colony-forming units and non-viable probiotic cell-equivalents. The probiotic may be naturally-occurring, synthetic, or developed through the genetic manipulation of organisms, whether such source is now known or later developed.

The composition may comprise a viable probiotic in the range of about 1×10⁴ colony forming units per 100 kilocalories (CFU/100 kcal) to about 1.5×10¹² CFU/100 kcal. Preferably, the composition comprises a viable probiotic in the range of about 1×10⁶ CFU/100 kcal to about 1×10⁹ CFU/100 kcal. More preferably, the composition comprises a viable probiotic in the range of about 1×10⁷ CFU/100 kcal to about 1×10⁸ CFU/100 kcal.

The composition may comprise one or more prebiotics. The prebiotic may comprise oligosaccharides, polysaccharides, or any other prebiotics that comprise fructose, xylose, soya, galactose, glucose, mannose, or any combination thereof. More specifically, the prebiotic may comprise polydextrose (PDX), polydextrose powder, lactulose, lactosucrose, raffinose, glucooligosaccharides, inulin, fructooligosaccharides, isomaltooligosaccharides, soybean oligosaccharides, lactosucrose, xylooligosaccharides, chitooligosaccharides, mannooligosaccharides, aribino-oligosaccharides, sialyloligosaccharides, fucooligosaccharides, galactooligosaccharides (GOS), and gentiooligosaccharides.

The composition may comprise a prebiotic in the range of about 1.0 g/L to about 10.0 g/L of the composition. Preferably, the composition comprises a prebiotic in the range of about 2.0 g/L and about 8.0 g/L of the composition. Alternatively, the composition may comprise a prebiotic in the range of about 0.01 g/100 kcal to about 1.5 g/100 kcal. Preferably, the composition comprises a prebiotic in the range of about 0.15 g/100 kcal to about 1.5 g/100 kcal.

The composition may comprise a prebiotic comprising PDX, GOS, or a combination thereof.

The composition may comprise PDX in the range of about 1.0 g/L and 10.0 g/L. Preferably, the composition comprises PDX in the range of about 2.0 g/L and 8.0 g/L. Alternatively, the composition comprises PDX in the range of about 0.015 g/100 kcal to about 1.5 g/100 kcal. Preferably, the composition comprises PDX in the range of about 0.05 g/100 kcal to about 1.5 g/100 kcal. More preferably, the composition comprises PDX in the range of about 0.2 g/100 kcal to about 0.6 g/100 kcal.

The composition may comprise GOS in the range of about 0.015 g/100 kcal to about 1.0 g/100 kcal. Preferably, the composition comprises GOS in the range of about 0.2 g/100 kcal to about 0.5 g/100 kcal.

The composition may comprise PDX in combination with GOS. Advantageously, the combination of PDX and GOS may stimulate and/or enhance endogenous butyrate production by microbiota. The composition may comprise GOS and PDX in a total amount of at least about 0.015 g/100 kcal. The composition may comprise GOS and PDX in a total amount in the range of about 0.015 g/100 kcal to about 1.5 g/100 kcal. Preferably, the composition comprises GOS and PDX in a total amount in the range of about 0.1 g/100 kcal to about 1.0 g/100 kcal. The prebiotic may comprise at least 20% weight per weight (w/w) PDX, GOS, or a combination thereof.

The composition may comprise lactoferrin. Lactoferrin may be present in the composition in an amount of at least about 15 mg/100 kcal. The composition may comprise lactoferrin in the range of about 15 mg/100 kcal to about 25 g/100 kcal. Preferably, the composition comprises lactoferrin in the range of about 5 g/100 kcal to about 20 g/100 kcal. More preferably, the composition comprises lactoferrin in the range of about 10 g/100 kcal to about 15 g/100 kcal. The composition may be specifically designed for a paediatric subject. When the composition is specifically intended for a paediatric subject, the composition may comprise lactoferrin in the range of about 15 mg/100 kcal to about 300 mg/100 kcal. Preferably, the composition comprises lactoferrin in the range of about 60 mg to about 150 mg/100 kcal. More preferably, the composition comprises lactoferrin in the range of about 60 mg/100 kcal to about 100 mg/100 kcal.

The composition may comprise one or more human milk oligosaccharides (HMOs). The HMO may comprise 2′-fucosyllactose (2FL), 3′-fucosyllactose (3FL), lacto-N-tetraose (LNT), lacto-N-neotetraose (LNnT), lacto-N-fucopentaose I (LNFP-I), 3′-sialyllactose (3SL), 6′-sialyllactose (6SL), or a combination thereof.

The composition may comprise an HMO in the range of about 0.01 g/L to about 5.0 g/L. Preferably, the composition comprises an HMO in the range of about 0.05 g/L to about 4.0 g/L of the composition. More preferably, the composition comprises an HMO in the range of about 0.05 g/L to about 2.0 g/L of the composition. Alternatively, the composition may comprise an HMO in the range of about 0.01 g/100 kcal to about 2.0 g/100 kcal. Preferably, the composition comprises an HMO in the range of about 0.01 g/100 kcal to about 1.5 g/100 kcal.

The composition may comprise a source of long-chain polyunsaturated fatty acids (LCPUFAs). The source of LCPUFAs may comprise docosahexaenoic acid (DHA), α-linoleic acid, γ-linoleic acid, linoleic acid, linolenic acid, eicosapentaenoic acid (EPA), arachidonic acid (ARA), or any combination thereof. Preferably, the composition comprises a source of LCPUFAs comprising DHA, ARA, or a combination thereof.

The composition may comprise an LCPUFA in an amount of at least about 5 mg/100 kcal. The composition may comprise an LCPUFA in the range of about 5 mg/100 kcal to about 100 mg/100 kcal. Preferably, the composition comprises an LCPUFA in the range of about 10 mg/100 kcal to about 50 mg/100 kcal.

The composition may comprise DHA in the range of about 5 mg/100 kcal to about 80 mg/100 kcal. Preferably, the composition comprises DHA in the range of about 10 mg/100 kcal to about 20 mg/100 kcal. More preferably, the composition comprises DHA in the range of about 15 mg/100 kcal to about 20 mg/100 kcal.

The composition may comprise ARA in the range of about 10 mg/100 kcal to about 100 mg/100 kcal of ARA. Preferably, the composition comprises ARA in the range of about 15 mg/100 kcal to about 70 mg/100 kcal. More preferably, the composition comprises ARA in the range of about 20 mg/100 kcal to about 40 mg/100 kcal.

The composition may comprise both DHA and ARA. The weight ratio of ARA:DHA may be in the range of about 1:3 to about 9:1. Preferably, the weight ratio of ARA:DHA is in the range of about 1:2 to about 4:1. The composition may comprise oils containing DHA and/or ARA. If utilised, the source of DHA and/or ARA may be any source known in the art such as marine oil, fish oil, single cell oil, egg yolk lipid, or brain lipid. The DHA and ARA may be sourced from single cell oils, DHASCOR and ARASCOR from DSM Nutritional Products, or variations thereof. The DHA and ARA may be in a natural form, provided that the remainder of the LCPUFA source does not result in any substantial deleterious effect on the subject. Alternatively, the DHA and ARA may be used in refined form.

The composition may comprise β-glucan. Preferably, the β-glucan comprises β-1,3-glucan. Preferably, the β-1,3-glucan comprises β-1,3;1,6-glucan. The composition may comprise β-glucan present in the range of about 0.010 grams to about 0.080 grams per 100 g of composition. Alternatively, the composition may comprise ß-glucan in the range of about 3 mg/100 kcal to about 17 mg/100 kcal. Preferably, the composition comprises β-glucan in the range of about 4 mg/100 kcal to about 17 mg/100 kcal.

The composition may comprise sialic acid. Mammalian brain tissue contains the highest levels of sialic acid as sialic acid is incorporated into brain-specific proteins, such as the neural cell adhesion molecule (NCAM) and lipids (e.g. gangliosides). Sialic acid is therefore believed to play an important role in neural development and function, learning, cognition, and memory.

The composition may comprise sialic acid provided by an inherent source (such as eWPC), exogenous sialic acid, sialic acid from sources (such as cGMP), or any combination thereof. The composition may comprise sialic acid in the range of about 100 mg/L to about 800 mg/L. Preferably, the composition comprises sialic acid in the range of about 120 mg/L to about 600 mg/L. More preferably, the composition comprises sialic acid in the range of about 140 mg/L to about 500 mg/L. Alternatively, the composition may comprise sialic acid in the range of about 1 mg/100 kcal to about 120 mg/100 kcal. Preferably, the composition comprises sialic acid in the range of about 14 mg/100 kcal to about 90 mg/100 kcal. More preferably, the composition comprises sialic acid in the range of about 15 mg/100 kcal to about 75 mg/100 kcal.

The composition may comprise one or more suitable composition ingredient, wherein the suitable composition ingredient comprises choline, inositol, an emulsifier, a preservative, a stabiliser, or a combination thereof. The composition may comprise choline. Choline is a nutrient that is essential for normal function of cells. Choline is a precursor for membrane phospholipids and it accelerates the synthesis and release of acetylcholine, a neurotransmitter involved in memory storage. Without wishing to be bound by theory, it is believed that dietary choline and docosahexaenoic acid (DHA) act synergistically to promote the biosynthesis of phosphatidylcholine and thus, help promote synaptogenesis in human subjects. Additionally, choline and DHA act synergistically to promote dendritic spine formation, which is important in the maintenance of established synaptic connections. The composition may comprise about 20 mg to about 100 mg of choline per 8 fl. oz. (236.6 mL) serving.

The composition may comprise inositol. The inositol may be present as exogenous inositol, inherent inositol, or a combination thereof. The composition may comprise inositol in the range of about 10 mg/100 kcal to 40 mg/100 kcal. Preferably, the composition comprises inositol in the range of about 20 mg/100 kcal to 40 mg/100 kcal. Alternatively, the composition comprises inositol in the range of about 130 mg/L to about 300 mg/L.

The composition may comprise one or more emulsifier, as an emulsifier can increase the stability of the composition. The emulsifier may comprise, but is not limited to, egg lecithin, soy lecithin, alpha lactalbumin, monoglycerides, diglycerides, or any combination thereof.

The composition may comprise one or more preservative, as a preservative can extend the shelf-life of the composition. The preservative may comprise, but is not limited to, potassium sorbate, sodium sorbate, potassium benzoate, sodium benzoate, calcium disodium EDTA, or any combination thereof.

The composition may comprise one or more stabiliser, as a stabiliser can help preserve the structure of the composition. The stabiliser may comprise, but is not limited to, gum arabic, gum ghatti, gum karaya, gum tragacanth, agar, furcellaran, guar gum, gellan gum, locust bean gum, pectin, low methoxyl pectin, gelatine, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, or any combination thereof.

The composition may be intended for a paediatric subject or an adult subject. The paediatric subject may be an infant or a child. The infant may be a full-term infant or a preterm infant. Preferably, the infant is a pre-term infant. The infant may be a vaginally-delivered infant. Alternatively, the infant may be an infant delivered by C-section. The gut microbiota play a significant role in the development and maturation of the immune system. It is known that the gut microbiota of C-section infants is different to infants that were vaginally delivered, with a study showing that C-section birth is associated with an increased likelihood of immune and metabolic disorders such as allergies, asthma, hypertension, and obesity (Hansen et al., J Immunol Aug. 1, 2014, 193 (3) 1213-1222). One possible way of reducing the likelihood of immune and metabolic disorders in C-section infants may be the provision of a composition comprising lactoferrin and/or beneficial probiotics such as LGG and B. infantis, in an attempt to bring the gut microbiota of the C-section infants into closer alignment with the gut microbiota of vaginally-delivered infants.

The composition may be a nutritional composition. The nutritional composition may be a nutritional supplement, a children's nutritional product, an infant formula, a human milk fortifier, a follow-up formula, a young child milk, or any other composition designed for a paediatric subject. Preferably, the composition is an infant formula or a human milk fortifier. More preferably, the composition is a preterm infant formula or a human milk fortifier.

The composition may be provided in an orally-ingestible form, wherein the orally-ingestible comprises a food, a beverage, a tablet, a capsule, or a powder. The composition may be expelled directly into a subject's intestinal tract. The composition may be expelled directly into the gut. The composition may be formulated to be consumed or administered enterally under the supervision of a physician.

The composition may be suitable for a number of dietary requirements. The composition may be kosher. The composition may be a non-genetically modified product. The composition may be sucrose-free. The composition may also be lactose-free. The composition may not contain any medium-chain triglyceride oil. No carrageenan may be present in the composition. The composition may be free of all gums.

The scope of the present invention is defined in the appended claims. It is to be understood that the skilled person may make amendments to the scope of the claims without departing from the scope of the present disclosure.

### DESCRIPTION OF FIGURES

FIG. 1: A plot showing the effect of extensively hydrolysed casein on cytokine expression (IL-1β (FIG. 1a) and IL-8 (FIG. 1b)) under baseline conditions in an in vitro foetal epithelial cell model.

FIG. 2: A plot showing the effect of extensively hydrolysed casein on cytokine expression (TNF) under baseline conditions in an in vitro foetal epithelial cell model.

FIG. 3: A plot showing the effect of extensively hydrolysed casein on foetal intestinal permeability under baseline conditions in an in vitro foetal epithelial cell model.

FIG. 4: A plot showing the effect of the exposure of foetal intestinal epithelium to heat-killed commensal E. coli on cytotoxicity in the absence (FIG. 4a) or presence (FIG. 4b) of extensively hydrolysed casein.

FIG. 5: A plot showing the effect of the exposure of foetal intestinal epithelium to heat-killed commensal E. coli on the expression of tumour necrosis factor receptor 1 (TNFR1; FIG. 5a) or myosin light chain kinase (MLCK; FIG. 5b), in the absence or presence of extensively hydrolysed casein.

FIG. 6: A plot showing the effect of the exposure of foetal intestinal epithelium (FIG. 6a) or adult intestinal epithelium (FIG. 6b) to heat-killed commensal E. coli on the expression of tumour necrosis factor receptor 2 (TNFR2), in the absence of extensively hydrolysed casein.

FIG. 7: A plot showing the effect of the exposure of foetal intestinal epithelium to heat-killed commensal E. coli on the expression of TNFR2, in the absence or presence of extensively hydrolysed casein.

FIG. 8: A plot showing the effect of the exposure of foetal intestinal epithelium to a TNF/IFNγ-induced proinflammatory environment on cell viability, in the absence or presence of extensively hydrolysed casein.

### EXPERIMENTAL PROCEDURE

To investigate the effect of extensively hydrolysed protein on preterm infant gut barrier function and integrity, and innate immune response in preterm infants, an in vitro human-derived epithelial monolayer culture was utilised as an intestinal epithelial model. Foetal duodenal-derived primary enteroids, representative of premature intestinal epithelium, were employed in the intestinal epithelial model. The model therefore facilitates physiologically relevant testing of the potential protective effects of functional infant formula ingredients on an immature developing intestine i.e. a preterm infant gut.

The foetal enteroids were cultured and plated on a transwell format to generate epithelial monolayers as previously described (van Dussen et al., 2015; Senger et al., 2018). The foetal epithelial monolayers were exposed to extensively hydrolysed casein (at 10 micrograms per millilitre (μg/mL), 100 μg/mL, and 1000 μg/mL) under baseline (Study 1) or challenged (Study 2) conditions, relative to a non-exposed ‘control’ monolayer, with each individual experiment repeated at least in triplicate.

### Study 1: Effects of Exposure of Foetal Epithelial Monolayer to Extensively Hydrolysed Casein Under Baseline Conditions

The foetal epithelial monolayer was incubated for 24 hours in the absence or presence of extensively hydrolysed casein, with the effect of the presence of extensively hydrolysed casein on cytokine expression and foetal epithelial monolayer permeability assessed.

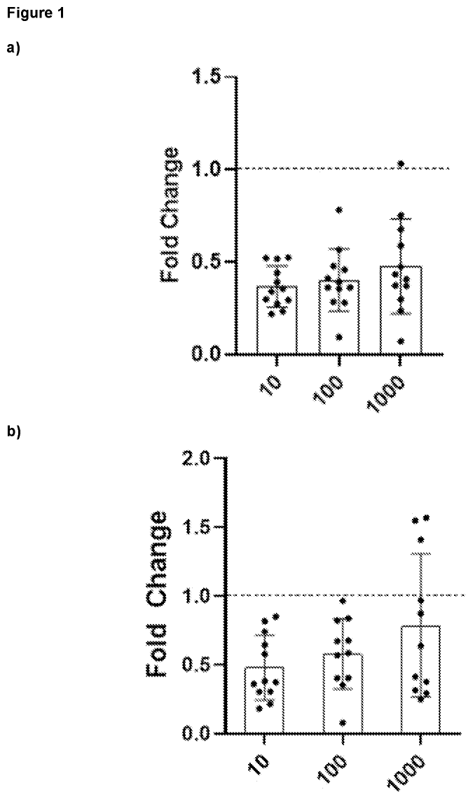

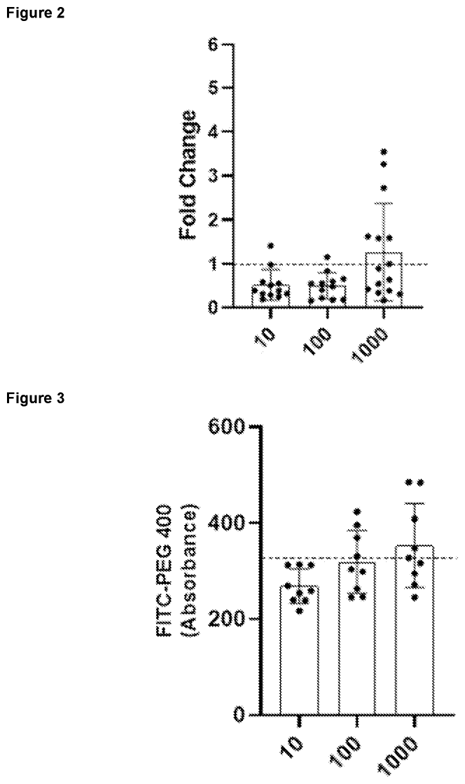

Cytokine expression, upon exposure to extensively hydrolysed casein, was analysed and quantified by Real-Time quantitative Reverse Transcription Polymerase Chain Reaction (qRT-PCR). As shown in FIG. 1, incubation of the monolayer with extensively hydrolysed casein decreased the expression of IL-1β and IL-8 at all concentrations of extensively hydrolysed casein. A similar downregulation effect was seen with respect to TNF expression at 10 μg/mL and 100 μg/mL of extensively hydrolysed casein (see FIG. 2). Cytokines such as IL-1β, IL-8, and TNF are indicators of inflammation and, in certain cell contexts, act as pro-inflammatory mediators. This data therefore suggests that extensively hydrolysed casein may be able to reduce, or even prevent, intestinal inflammation in a preterm infant.

Transepithelial passage of PEG 400-FITC were employed to evaluate changes in foetal epithelial monolayer permeability upon exposure to extensively hydrolysed casein. As can be seen in FIG. 3, incubation with 10 μg/mL of extensively hydrolysed casein significantly reduced the paracellular permeability of the foetal intestinal epithelium, when compared to the control (shown as a horizontal dashed line) i.e. the presence of 10 μg/mL extensively hydrolysed casein strengthened the foetal intestinal epithelium and made it less susceptible to paracellular passage. This result demonstrates that extensively hydrolysed casein is able to improve preterm infant gut barrier integrity, and thus, indicates that extensively hydrolysed casein could improve preterm infant gut barrier function.

In summary, the results from the baseline experiments suggest that extensively hydrolysed casein may be useful in reducing intestinal inflammation and/or improving gut barrier function, in a preterm infant.

### Study 2: Effects of Exposure of Foetal Epithelial Monolayer to Extensively Hydrolysed Casein Under Challenged Conditions

Study 2a: Foetal Epithelial Monolayer Challenged with Heat-Killed Commensal E. coli

The foetal epithelial monolayer was incubated for 24 hours in the absence or presence of extensively hydrolysed casein, prior to being exposed to heat-killed commensal non-pathogenic human isolate E. coli bacteria (hereinafter “heat-killed commensal E. coli”) for four hours.

The cytotoxicity to the foetal epithelial monolayer induced by the presence of heat-killed commensal E. coli, in the absence or presence of extensively hydrolysed casein, was evaluated using the commercially available CytoTox 96® lactate dehydrogenase (LDH) assay kit (Promega).

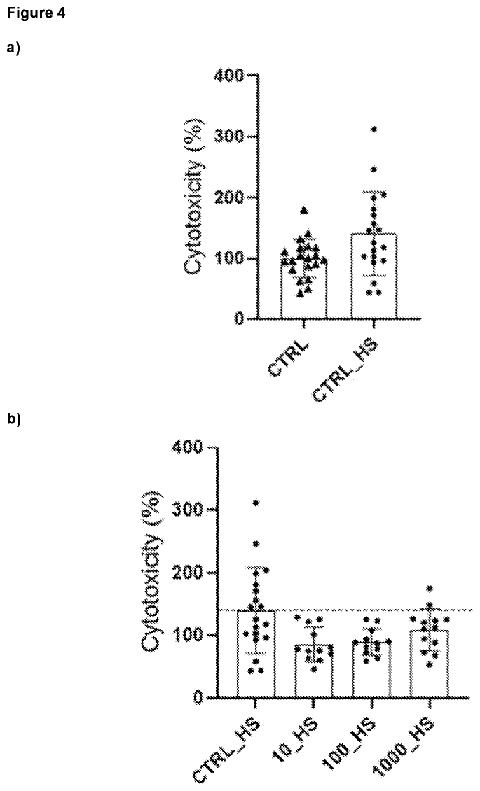

As can be seen in FIG. 4a, foetal epithelial cytotoxicity substantially increased when the foetal epithelial monolayer was exposed to heat-killed commensal E. coli. The foetal epithelial cytotoxicity was substantially reduced when the foetal intestinal epithelium was previously incubated with extensively hydrolysed casein (see FIG. 4b) i.e. extensively hydrolysed casein substantially improved foetal epithelium cell viability in response to heat-killed commensal E. coli exposure. This result shows that extensively hydrolysed casein is able to exert a protective effect on the preterm infant gut, in response to the presence of heat-killed commensal E. coli, and indicates that extensively hydrolysed casein may improve preterm infant gut barrier integrity and/or reduce the risk of intestinal tissue damage.

The nature and mechanism of the observed protective effect of extensively hydrolysed casein was then explored. As mentioned previously, TNF can act as a negative regulator of tight junctions via one of two distinct pathways: (i) downregulation of zonula occludens-1 (ZO1) and/or occludin; or, (ii) modulation of MLCK expression via interaction with TNFR1 and/or TNFR2. The experimental results indicated that the observed protective effect of extensively hydrolysed casein did not proceed via route (i), as extensively hydrolysed casein didn't have any effect on ZO1 or occludin expression (data not shown), so route (ii) was then investigated.

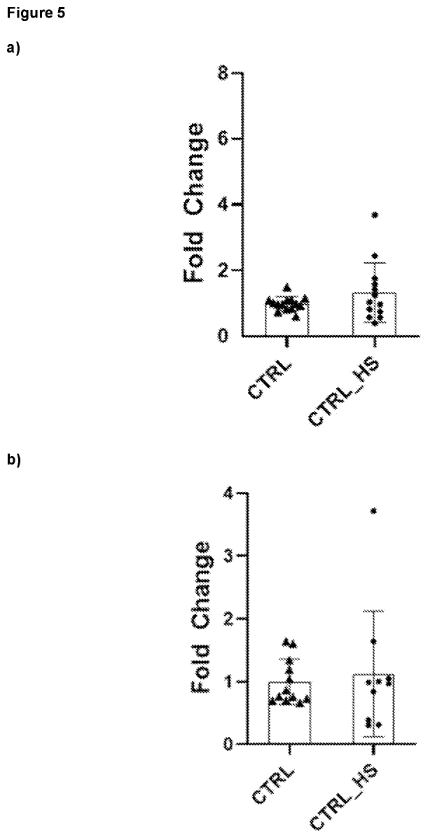

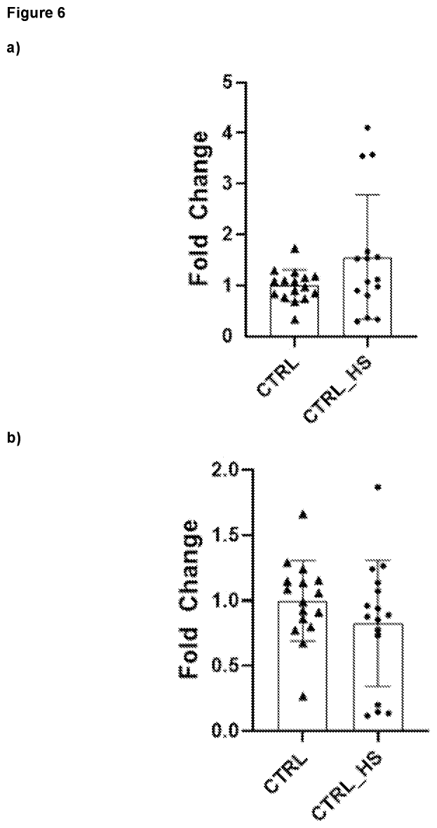

The effect of the exposure of foetal intestinal epithelium to heat-killed commensal E. coli on the expression of the TNFR1, TNFR2, and MLCK, in the absence of extensively hydrolysed casein, was assessed using qRT-PCR. Exposure of the foetal epithelial monolayer to heat-killed commensal E. coli had no significant effect on the expression of TNFR1 (FIG. 5a) or MLCK (FIG. 5b), but upregulated expression of TNFR2 (FIG. 6a).

To investigate whether the observed upregulation of TFNR2 expression was specific to preterm infant epithelium, an adult epithelial monolayer was prepared via the same process as for the foetal epithelial monolayer, but using adult duodenal-derived primary enteroids, and subjected to the same experimental conditions. Interestingly, it was found that the presence of heat-killed commensal E. coli, in the absence of extensively hydrolysed casein, had no significant effect on TNRF2 expression in adult epithelium (see FIG. 6b). This result may partly explain why preterm infant gut reacts differently to the presence of heat-killed commensal E. coli, compared to full-term infants and adults.

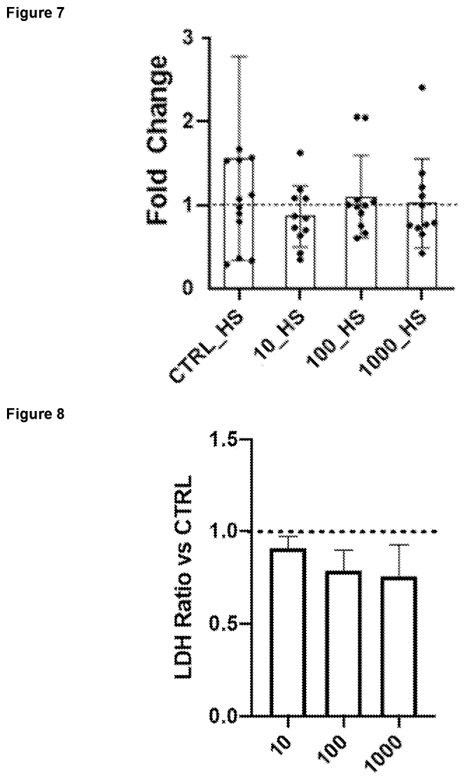

To see if the observed protective effect of extensively hydrolysed casein was due to an effect on TNFR2 expression, the effect of exposure of foetal intestinal epithelium to heat-killed commensal E. coli on the expression of TNFR2, in the absence (control) or presence of extensively hydrolysed casein, was assessed using qRT-PCR. As FIG. 7 shows, all concentrations of extensively hydrolysed casein significantly downregulated TNFR2 expression, when compared to the control.

In summary, the results of Study 2a suggest that extensively hydrolysed casein is able to exert a protective effect on foetal epithelium, when exposed to heat-killed commensal E. coli, as it increases foetal epithelial cell viability, and that this protective effect may occur through downregulation of TNFR2 expression and the consequent quenching of the negative effects of TNF on MLCK expression and actin cytoskeleton structure.

Study 2b: Foetal Epithelial Monolayer Challenged with a Pro-Inflammatory Environment

The foetal epithelial monolayer was incubated for 24 hours in the absence or presence of extensively hydrolysed casein, prior to being exposed to a pro-inflammatory environment created by incubation of the foetal epithelial monolayer in the presence of 25 ng/ml TNF and 0.1 ng/ml interferon-γ (IFN-γ) for 24 hours. As with Study 1, the cytotoxicity of the foetal epithelial monolayer was evaluated using the commercially available CytoTox 96® LDH assay kit.

As can be seen in FIG. 8, incubation with extensively hydrolysed casein significantly reduced the foetal epithelial cytotoxicity at all concentrations i.e. extensively hydrolysed casein improved foetal epithelium cell viability in response to pro-inflammatory environment exposure. This result shows that extensively hydrolysed casein is able to exert a protective effect on the preterm infant gut, in response to the presence of a pro-inflammatory environment, and indicates that extensively hydrolysed casein may improve preterm infant gut barrier integrity and/or reduce the risk of intestinal tissue damage, in a challenging environment.

### Example Compositions

The compositions shown in Table 2 illustrate examples of compositions within the scope of the present disclosure, but are in no way intended to provide any limitation on the disclosure.

TABLE 2

| | Composition | | | | | | | | | |
|---|---|---|---|---|---|---|---|---|---|---|
| Component | I | II | III | IV | V | VI | VII | VIII | IX | X |
| Extensively hydrolysed | ✓ | ✓ | ✓ | ✓ | ✓ | ✓ | ✓ | ✓ | ✓ | ✓ |
| protein (such as extensively | | | | | | | | | | |
| hydrolysed casein) | | | | | | | | | | |
| LGG | X | ✓ | X | X | X | X | X | X | X | X |
| B. infantis | X | X | ✓ | X | X | X | X | X | X | X |
| Enriched milk product | X | X | X | ✓ | X | X | X | X | X | X |
| PDX | X | X | X | X | ✓ | ✓ | X | X | X | X |
| GOS | X | X | X | X | X | ✓ | X | X | X | X |
| DHA | X | X | X | X | X | X | ✓ | ✓ | X | X |
| ARA | X | X | X | X | X | X | X | ✓ | X | X |
| Lactoferrin | X | X | X | X | X | X | X | X | ✓ | X |
| HMO | X | X | X | X | X | X | X | X | X | ✓ |

| | Composition | | | | |
|---|---|---|---|---|---|
| Component | XI | XII | XIII | XIV | XV |
| Extensively hydrolysed | ✓ | ✓ | ✓ | ✓ | ✓ |
| protein (such extensively | | | | | |
| hydrolysed casein) | | | | | |
| LGG | ✓ | ✓ | X | X | ✓ |
| B. infantis | X | X | ✓ | ✓ | ✓ |
| Enriched milk product | ✓ | ✓ | ✓ | ✓ | ✓ |
| PDX | ✓ | ✓ | ✓ | ✓ | ✓ |
| GOS | ✓ | ✓ | ✓ | ✓ | ✓ |
| DHA | ✓ | ✓ | ✓ | ✓ | ✓ |
| ARA | ✓ | ✓ | ✓ | ✓ | ✓ |
| Lactoferrin | ✓ | ✓ | ✓ | ✓ | ✓ |
| HMO | X | ✓ | X | ✓ | ✓ |

Key:
✓ = present;
X = not present

1. Extensively hydrolysed casein for use in reducing the risk of intestinal inflammation, intestinal tissue damage, or both, in a preterm infant.

2. The extensively hydrolysed casein for use according to claim 1, wherein reducing the risk of intestinal inflammation, intestinal tissue damage, or both comprises a decrease in the expression of one or more of IL-1β, IL-8, and TNFR2, in the intestinal epithelium of the preterm infant, by 1% to 100%, compared to the absence of extensively hydrolysed casein.

3. The extensively hydrolysed casein for use according to claim 1 or 2, wherein reducing the risk of intestinal inflammation, intestinal tissue damage, or both, in a preterm infant comprises reducing the risk of necrotising enterocolitis (NEC), systemic infection, or both.

4. Extensively hydrolysed casein for use in improving gut barrier function in a preterm infant.

5. The extensively hydrolysed casein for use according to claim 4, wherein improving gut barrier function comprises improving intestinal cell viability in the preterm infant.

6. The extensively hydrolysed casein for use according to claim 4, wherein improving gut barrier function comprises improving gut barrier integrity in the preterm infant.

7. The extensively hydrolysed casein for use according to claim 6, wherein improving gut barrier integrity comprises an increase in gut barrier integrity of 1% to 100%, an increase in intestinal cell viability of 1% to 100%, or both, compared to the absence of extensively hydrolysed casein.

8. The extensively hydrolysed casein for use according to claim 6 or 7, wherein improving gut barrier integrity comprises reducing intestinal permeability in the preterm infant.

9. The extensively hydrolysed casein for use according to claim 8, wherein reducing intestinal permeability comprises a decrease in gut barrier permeability of 1% to 100%, compared to the absence of extensively hydrolysed casein.

10. The extensively hydrolysed casein for use according to claim 6 or 7, wherein improving gut barrier integrity comprises improving intestinal cell viability in the preterm infant.

11. The extensively hydrolysed casein for use according to claim 10, wherein improving intestinal cell viability comprises an increase in intestinal cell viability of 1% to 100%, compared to the absence of extensively hydrolysed casein.

12. Extensively hydrolysed casein for use in downregulating expression of IL-1β, IL-8, TNFR2, or any combination thereof, in a preterm infant.

13. The extensively hydrolysed casein for use according to claim 12, wherein downregulating expression of IL-1β, IL-8, TNFR2, or any combination thereof comprises a decrease in the expression of one or more of IL-1β, IL-8, and TNFR2, in the intestinal epithelium of the preterm infant, by 1% to 100%, compared to the absence of extensively hydrolysed casein.

14. The extensively hydrolysed casein for use according to any of claims 1 to 13, wherein the extensively hydrolysed casein comprises at least three peptides selected from SEQ ID NO: 1 to SEQ ID NO: 68.

15. The extensively hydrolysed casein for use according to claim 14, wherein the extensively hydrolysed casein comprises at least three peptides selected from SEQ ID NO: 1 to SEQ ID NO: 12.

16. The extensively hydrolysed casein for use according to any of claims 1 to 15, wherein the extensively hydrolysed casein is in the form of a reconstituted solution, preferably wherein the reconstituted solution comprises extensively hydrolysed casein in the range of about 0.01 milligrams per millilitre (mg/ml) to about 0.50 grams per millilitre (g/mL).

17. A composition for use in: reducing the risk of intestinal inflammation, intestinal tissue damage, or both; improving gut barrier function; and/or, downregulating expression of IL-1β, IL-8, TNFR2, or any combination thereof, in a preterm infant, wherein the composition comprises extensively hydrolysed casein.

18. The composition for use according to claim 17, wherein the extensively hydrolysed casein comprises at least three peptides selected from SEQ ID NO: 1 to SEQ ID NO: 68.

19. The composition for use according to claim 18, wherein the extensively hydrolysed casein comprises at least three peptides selected from SEQ ID NO: 1 to SEQ ID NO: 12.

20. The composition for use according to any of claims 17 to 19, wherein the composition is in the form of a reconstitutable powder, preferably wherein the reconstitutable powder comprises extensively hydrolysed casein in the range of about 10 micrograms per 100 kilocalories (μg/100 kcal) to about 15 grams per 100 kilocalories (g/100 kcal).

21. The composition for use according to any of claims 17 to 20, wherein the composition further comprises at least one prebiotic, preferably the at least one prebiotic comprises polydextrose, galactooligosaccharides, or a combination thereof.

22. The composition for use according to any of claims 17 to 21, wherein the composition further comprises milk fat globule membrane (MFGM), preferably the MFGM is provided by an enriched milk product.

23. The composition for use according to any of claims 17 to 22, wherein the composition is a nutritional composition.

24. The composition for use according to any of claims 17 to 23, wherein the composition is a preterm infant formula or human milk fortifier.

25. The composition for use according to any of claims 17 to 24, wherein the composition is a synthetic composition.

26. A method for reducing the risk of intestinal inflammation, intestinal tissue damage, or both, in a preterm infant, the method comprising the step of administering extensively hydrolysed casein to the preterm infant.

27. The method of claim 26, wherein reducing the risk of intestinal inflammation, intestinal tissue damage, or both comprises a decrease in the expression of one or more of IL-1β, IL-8, and TNFR2, in the intestinal epithelium of the preterm infant, by 1% to 100%, compared to the absence of extensively hydrolysed casein.

28. The method of claim 26 or 27, wherein reducing the risk of intestinal inflammation, intestinal tissue damage, or both, in a preterm infant comprises reducing the risk of necrotising enterocolitis (NEC), systemic infection, or both.

29. A method for improving gut barrier function in a preterm infant the method comprising the step of administering extensively hydrolysed casein to the preterm infant.

30. The method of claim 29, wherein improving gut barrier function comprises improving intestinal cell viability in the preterm infant.

31. The method of claim 29, wherein improving gut barrier function comprises improving gut barrier integrity in the preterm infant.

32. The method of claim 31, wherein improving gut barrier integrity comprises an increase in gut barrier integrity of 1% to 100%, an increase in intestinal cell viability of 1% to 100%, or both, compared to the absence of extensively hydrolysed casein.

33. The method of claim 31 or 32, wherein improving gut barrier integrity comprises reducing intestinal permeability in the preterm infant.

34. The method of claim 33, wherein reducing intestinal permeability comprises a decrease in gut barrier permeability of 1% to 100%, compared to the absence of extensively hydrolysed casein.

35. The method of claim 31 or 32, wherein improving gut barrier integrity comprises improving intestinal cell viability in the preterm infant.

36. The method of claim 35, wherein improving intestinal cell viability comprises an increase in intestinal cell viability of 1% to 100%, compared to the absence of extensively hydrolysed casein.

37. A method for downregulating expression of IL-1β, IL-8, TNFR2, or any combination thereof, in a preterm infant, the method comprising the step of administering extensively hydrolysed casein to the preterm infant.

38. The method of claim 37, wherein downregulating expression of IL-1β, IL-8, TNFR2, or any combination thereof comprises a decrease in the expression of one or more of IL-1β, IL-8, and TNFR2, in the intestinal epithelium of the preterm infant, by 1% to 100%, compared to the absence of extensively hydrolysed casein.

39. The method of any of claims 26 to 38, wherein the extensively hydrolysed casein comprises at least three peptides selected from SEQ ID NO: 1 to SEQ ID NO: 68.

40. The method of claim 39, wherein the extensively hydrolysed casein comprises at least three peptides selected from SEQ ID NO: 1 to SEQ ID NO: 12.

41. The method of any of claims 26 to 40, wherein the extensively hydrolysed casein is in the form of a reconstituted solution, preferably wherein the reconstituted solution comprises extensively hydrolysed casein in the range of about 0.01 milligrams per millilitre (mg/mL) to about 0.50 grams per millilitre (g/mL).

42. A method for: reducing the risk of intestinal inflammation, intestinal tissue damage, or both; improving gut barrier function; and/or, downregulating expression of IL-1β, IL-8, TNFR2, or any combination thereof, in a preterm infant, the method comprising administering a composition comprising extensively hydrolysed casein to the preterm infant.

43. The method of claim 42, wherein the extensively hydrolysed casein comprises at least three peptides selected from SEQ ID NO: 1 to SEQ ID NO: 68.

44. The method of claim 43, wherein the extensively hydrolysed casein comprises at least three peptides selected from SEQ ID NO: 1 to SEQ ID NO: 12.

45. The method of any of claims 42 to 44, wherein the composition is in the form of a reconstitutable powder, preferably wherein the reconstitutable powder comprises extensively hydrolysed casein in the range of about 10 micrograms per 100 kilocalories (μg/100 kcal) to about 15 grams per 100 kilocalories (g/100 kcal).

46. The method of any of claims 42 to 45, wherein the composition further comprises at least one prebiotic, preferably the at least one prebiotic comprises polydextrose, galactooligosaccharides, or a combination thereof.

47. The method of any of claims 42 to 46, wherein the composition further comprises milk fat globule membrane (MFGM), preferably the MFGM is provided by an enriched milk product.

48. The method of any of claims 42 to 47, wherein the composition is a nutritional composition.

49. The method of any of claims 42 to 48, wherein the composition is a preterm infant formula or human milk fortifier.

50. The method of any of claims 42 to 49, wherein the composition is a synthetic composition.


SEQUENCE LISTING (68 sequences)

SEQ ID NO: 1 - protein, 6 aa, BOVINE
IPNPIG

SEQ ID NO: 2 - protein, 9 aa, BOVINE
IGSESTEDQ

SEQ ID NO: 3 - protein, 7 aa, BOVINE
DKTEIPT

SEQ ID NO: 4 - protein, 4 aa, BOVINE
IVPN

SEQ ID NO: 5 - protein, 6 aa, BOVINE
LEDSPE

SEQ ID NO: 6 - protein, 6 aa, BOVINE
NQEQPI

SEQ ID NO: 7 - protein, 5 aa, BOVINE
NVPGE

SEQ ID NO: 8 - protein, 6 aa, BOVINE
PFPGPI

SEQ ID NO: 9 - protein, 5 aa, BOVINE
TEDEL

SEQ ID NO: 10 - protein, 4 aa, BOVINE
VPSE

SEQ ID NO: 11 - protein, 6 aa, BOVINE
YPFPGP

SEQ ID NO: 12 - protein, 5 aa, BOVINE
YPSGA

SEQ ID NO: 13 - protein, 6 aa, BOVINE
FPGPIP

SEQ ID NO: 14 - protein, 11 aa, BOVINE
MHQPHQPLPPT

SEQ ID NO: 15 - protein, 9 aa, BOVINE
YPFPGPIPN

SEQ ID NO: 16 - protein, 5 aa, BOVINE
DMEST

SEQ ID NO: 17 - protein, 7 aa, BOVINE
FPGPIPN

SEQ ID NO: 18 - protein, 5 aa, BOVINE
IPNPI

SEQ ID NO: 19 - protein, 6 aa, BOVINE
MESTEV

SEQ ID NO: 20 - protein, 6 aa, BOVINE
PGPIPN

SEQ ID NO: 21 - protein, 8 aa, BOVINE
PHQPLPPT

SEQ ID NO: 22 - protein, 4 aa, BOVINE
PNPI

SEQ ID NO: 23 - protein, 7 aa, BOVINE
SKDIGSE

SEQ ID NO: 24 - protein, 8 aa, BOVINE
YPFPGPIP

SEQ ID NO: 25 - protein, 8 aa, BOVINE
AINPSKEN

SEQ ID NO: 26 - protein, 5 aa, BOVINE
APFPE

SEQ ID NO: 27 - protein, 6 aa, BOVINE
DIGSES

SEQ ID NO: 28 - protein, 4 aa, BOVINE
DMPI

SEQ ID NO: 29 - protein, 4 aa, BOVINE
DVPS

SEQ ID NO: 30 - protein, 3 aa, BOVINE
EDI

SEQ ID NO: 31 - protein, 3 aa, BOVINE
ELF

SEQ ID NO: 32 - protein, 3 aa, BOVINE
EMP

SEQ ID NO: 33 - protein, 7 aa, BOVINE
ETAPVPL

SEQ ID NO: 34 - protein, 4 aa, BOVINE
GPFP

SEQ ID NO: 35 - protein, 4 aa, BOVINE
GPIV

SEQ ID NO: 36 - protein, 8 aa, BOVINE
IGSSSEES

SEQ ID NO: 37 - protein, 9 aa, BOVINE
IGSSSEESA

SEQ ID NO: 38 - protein, 6 aa, BOVINE
INPSKE

SEQ ID NO: 39 - protein, 9 aa, BOVINE
IPPLTQTPV

SEQ ID NO: 40 - protein, 4 aa, BOVINE
ITAP

SEQ ID NO: 41 - protein, 7 aa, BOVINE
KHQGLPQ

SEQ ID NO: 42 - protein, 5 aa, BOVINE
LDVTP

SEQ ID NO: 43 - protein, 5 aa, BOVINE
LPLPL

SEQ ID NO: 44 - protein, 5 aa, BOVINE
NAVPI

SEQ ID NO: 45 - protein, 5 aa, BOVINE
NEVEA

SEQ ID NO: 46 - protein, 3 aa, BOVINE
NLL

SEQ ID NO: 47 - protein, 5 aa, BOVINE
PITPT

SEQ ID NO: 48 - protein, 6 aa, BOVINE
PNSLPQ

SEQ ID NO: 49 - protein, 8 aa, BOVINE
PQLEIVPN

SEQ ID NO: 50 - protein, 7 aa, BOVINE
PQNIPPL

SEQ ID NO: 51 - protein, 6 aa, BOVINE
PVLGPV

SEQ ID NO: 52 - protein, 4 aa, BOVINE
PVPQ

SEQ ID NO: 53 - protein, 5 aa, BOVINE
PVVVP

SEQ ID NO: 54 - protein, 6 aa, BOVINE
PVVVPP

SEQ ID NO: 55 - protein, 11 aa, BOVINE
SIGSSSEESAE

SEQ ID NO: 56 - protein, 7 aa, BOVINE
SISSSEE

SEQ ID NO: 57 - protein, 11 aa, BOVINE
SISSSEEIVPN

SEQ ID NO: 58 - protein, 6 aa, BOVINE
SPPEIN

SEQ ID NO: 59 - protein, 7 aa, BOVINE
SPPEINT

SEQ ID NO: 60 - protein, 7 aa, BOVINE
TDAPSFS

SEQ ID NO: 61 - protein, 6 aa, BOVINE
VATEEV

SEQ ID NO: 62 - protein, 5 aa, BOVINE
VLPVP

SEQ ID NO: 63 - protein, 4 aa, BOVINE
VPGE

SEQ ID NO: 64 - protein, 6 aa, BOVINE
VPGEIV

SEQ ID NO: 65 - protein, 6 aa, BOVINE
VPITPT

SEQ ID NO: 66 - protein, 9 aa, BOVINE
VVPPFLQPE

SEQ ID NO: 67 - protein, 5 aa, BOVINE
VVVPP

SEQ ID NO: 68 - protein, 5 aa, BOVINE
YPVEP